(19) United States
(12) Patent Application Publication
(10) Pub. No.: US 2019/0192010 A1
(43) Pub. Date: Jun. 27, 2019

(54) DETECTION OF FLU USING THERMAL IMAGING

(71) Applicants: Viraj Mane, Toronto (CA); Syed Imtiaz Yakub, Pearland, TX (US)

(72) Inventors: Viraj Mane, Toronto (CA); Syed Imtiaz Yakub, Pearland, TX (US)

(21) Appl. No.: 16/225,793

(22) Filed: Dec. 19, 2018

Related U.S. Application Data
(60) Provisional application No. 62/609,558, filed on Dec. 22, 2017.

(51) Int. Cl. A61B 5/01; A61B 5/00; A61B 5/16; A61B 10/00; G16H 30/20; G16H 50/30

(52) U.S. Cl. CPC A61B 5/015; A61B 5/746; A61B 5/0022; A61B 5/7264; A61B 2010/0019; A61B 10/0012; A61B 5/6898; G16H 30/20; G16H 50/30; A61B 5/165

(57) ABSTRACT

Methods for determining change in physical condition or illness of a mammal by obtaining a thermal image of the subject and determining the body temperature of the mammal based on the thermal image are described. An example method is implemented on a first electronic device having a first display and a thermal imaging hardware. The method includes obtaining the thermal image of a mammal using the first electronic device; comparing said thermal image to a reference thermal image of the subject at healthy state with no symptoms or characteristics of the physical condition or illness such as by way of example, flu, fever, hypothermia, ovulation, heat stress, cardiac condition; comparing the intensity of said thermal image to the reference thermal image; and in response to such comparison, determining whether the subject shows symptoms or hallmarks of a certain illness or condition marked by a change in body temperature and associated intensity of the thermal image; and displaying information regarding said determination on the first display of the first electronic device. The method includes first obtaining a reference thermal image of a mammal in a “normal” and/or “healthy” condition, then later obtaining additional image(s) for comparison to the reference image; and in response to such comparison, determining whether the subject shows symptoms of a certain illness or condition marked by a change in body temperature and associated intensity of the thermal image.

### FIELD OF INVENTION

The field of the invention relates to determining the presence of influenza (flu) and other diseases or conditions with body temperature correlations in a locale, and extrapolating epidemiological data, by obtaining thermal imaging and associated data contained in user devices.

### BACKGROUND

### Infrared Measurement

Infrared is the next longest wavelength of light past visible light. Infrared wavelengths extend from approximately 700 nanometers (frequency 430 THz), to 1 millimeter (300 GHz). Currently, certain smartphone manufacturers use infrared emitters in their commercially-available smartphones (such as for example, iPhones from Apple Inc.) to project 30,000 infrared dots onto a user's face, capture the resulting infrared image, and compare it against reference images to verify the facial characteristics of the user in a biometric authentication protocol such as, for example, Face ID (Apple, 2018). In addition to this facial mapping application, however, it is well known that infrared sensors can also measure heat energy emitted (Wagner, 2013), and infrared cameras have been used for measurement of facial temperature (Ioannou, et al., 2014).

### Hardware

Infrared cameras have been proposed to correlate fever with influenza (flu) infection, notably as an airport screening measure (Priest, Duncan, Jennings, & Baker, 2011). However, this approach made use of stationary cameras that must rapidly scan multiple passengers and lacks a database to compare each image to a baseline reference for that specific person. Indeed, that study found a lack of predictive value in this approach.

As a contrast to that approach, we note that personal electronic devices such as smartphones, by way of example the iPhone with Face ID, are already configured to refer to the baseline of the device's owner, solving the aforementioned problem. As we will describe, assigning each device's analytical capabilities solely to its owner represents a novel computational approach at collecting individual health data that ultimately has community-level value.

### Machine Learning and Deep Learning

Machine learning is the exercise of using algorithms to train a computer to identify patterns in a dataset, in other words the automation of computational model-building. This exercise can be structured as supervised learning where the value of data inputs is known, for example, human temperature measurements as inputs can be ranked as “normal” or “pre-fever” or “fever” according to well-established clinical guidelines for fever. Machine learning can also be structured as unsupervised learning (data with no historical context), for example if it were trained to correlate fever with human commuter patterns where no such correlation was ever previously identified.

Deep learning can be thought of as pattern recognition (machine learning) from massive datasets. In contrast to the previous machine learning examples involving only one or two variables, a deep learning exercise could, by way of example, attempt to correlate fever against atmospheric temperature, humidity, weather patterns, season, etc. In addition to these environmental variables, the deep learning engine may also contemplate human factors such as commuter patterns, international travel patterns, and employment sector, all of which influence a person's likelihood of encountering influenza.

### Fever Monitoring (Individual Level)

Fever can be an early indicator of infection. Each influenza season, health organizations recommend that employees stay home and rest when sick rather than infecting their family, colleagues, and fellow commuters. In the 2017 flu season, more people were killed by seasonal influenza than in any other since the 1970s (Sun, 2017). However, there is no straightforward way for a person to self-diagnose the flu accurately when common flu symptoms may overlap across disparate conditions such as the common cold, allergy/asthma, hormonal imbalance, fatigue, etc. Conversely, fever is an objective measurement that helps narrow down this list of conditions. For example, during the common cold season among adults and children, fever is more likely to be caused by viral influenza as compared to a bacterial infection, and this guidance can be used to prevent unnecessary prescription of antibiotics (which are active against bacteria but not viruses).

Depending on geography, time of year, and travel history, fever is also a useful risk indicator for less common infections such as swine flu, Ebola, or Zika virus. Therefore there is utility in passive, frequent monitoring of body temperature because this can identify onset of fever which can be an objective, quantitative surrogate for early-stage infection.

However, passive and frequent temperature measurement is not possible with current tools. For example, rectal thermometers are highly accurate but extremely invasive and inconvenient. Conversely, axillary and tympanic temperature measurements are simple and fast, but axillary measurement often requires removal of clothing and tympanic measurement is susceptible to interference by earwax buildup. The development of infrared measurement at the forehead is an improvement in terms of speed and non-invasiveness of temperature collection. However, this is still a single-point collection, and the accuracy of forehead infrared measurement can be compromised by perspiration. More importantly, the forehead as a single measurement may not be a reliable surrogate for body temperature (Berksoy, Bağ, Yazici, & Çelik, 2018).

These examples underscore the limited value of single-point temperature measurements and the limitation of examining only a single region of the face or body. To this end, we describe a deep learning engine which creates a living record of an individual's temperature measurements over time, superimposing related external metadata (e.g. location, elevation, outdoor temperature, humidity, employment sector, proximity to mealtime or exercise routines) to further inform and refine a Fever Risk Assessment score that is ultimately returned to the individual. Providing clinical context to classify body temperature measurements as either “normal” or “pre-fever” or “fever” would be an example of “supervised learning”. This novel contextualizing of a person's body temperature measurement within up-to-date, high-level environmental and/or epidemiological frameworks deepens the value of this information for the individual user as well as for community health and global health professionals. Specifically, a person's interpretation of (and response to) fever would be improved if the person was made aware of patterns of infection within the local community, as described in the next section.

### Fever Monitoring (Community Level)

Influenza outbreaks occur one or more times every year. Epidemiological evidence suggests that flu outbreaks can originate in east Asian countries and then migrate westward. Because early-stage infection may be asymptomatic, the disease is unknowingly spread across communities, and travelers then spread it rapidly via mass transit such as trains and airplanes (which sequester contagious people in small cabins). In addition to influenza, various other pathogens have demonstrated rapid spread in the past few years, including Ebola, Zika, cholera, etc. The speed and magnitude of an outbreak can be exacerbated by various factors such as access to mass transit, weakness in healthcare infrastructure, weather patterns, lack of clean water infrastructure, consolidation of displaced people into refugee camps, etc. An important benefit of our proposed invention is that the diagnostic and epidemiological value of fever monitoring is agnostic and independent of the aforementioned exacerbating factors. Currently, there are mathematical modeling tools that account for common patterns of human transportation and movement that can be used to estimate the direction, speed, and severity of a disease outbreak. However, these models make use of historical data, internet search results, location-based news articles, and algorithmic estimates to develop predictions of future events. They suffer from a lack of up-to-date (real-time) information on the actual health and fever status of affected people.

We therefore describe the integration of individual user datapoints discussed in the previous section into a mapping database that would, for the first time, populate an epidemiological framework with actual human temperature measurements that are automatically meta-tagged with accurate time and location data. A deep learning system would collect individual-level information into large community-level datasets to identify real-time patterns of human congregation, travel, commuting, etc., and their relationship of these patterns to the incubation and spread of disease. Therefore, ongoing monitoring of fever would generate actionable human-level data that can also be aggregated into accurate models of disease spread at the hyperlocal, regional, or even global scale.

The results of this community-scale fever mapping tool are used to enrich the value of the risk assessment that is relayed back to individual users, as compared to an assessment of temperature on its own. Examples of the types of interaction with the invention, experienced by a representative individual named User A, are as follows:
User A has a history of being susceptible to seasonal flu (based on longitudinal data collected using the invention);
The epidemiological mapping tool observes that User A's typical commuter routes (based on geopositioning data correlated to bus and subway routes) have a high density of people with fever during the current flu season;
The mapping tool would report this multifactorial risk assessment to User A and would recommend alternative travel routes.

### BRIEF DESCRIPTION OF THE DRAWINGS

Reference will now be made, by way of example, to the accompanying drawings which show example embodiments of the present application and in which:

FIG. 1 is a diagram of a human face with numbers corresponding to universal mammalian facial regions;

FIG. 2 is a schematic of the invention's workflow as related to the flow of data.

### DETAILED DESCRIPTION OF EXAMPLE EMBODIMENTS

Example embodiments of the present disclosure will be apparent to those of ordinary skill in the art from a review of the following detailed descriptions in conjunction with the drawings.

Example embodiments of the present disclosure are not limited to any particular operating system, electronic device architecture, server architecture or computer programming language.

### Regions of a Mammalian Face

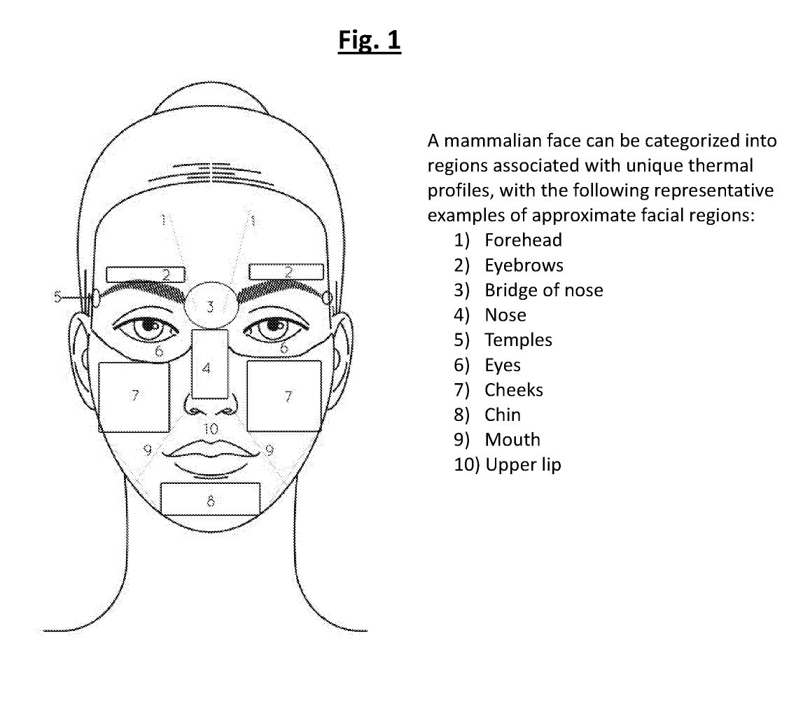

By reference to FIG. 1, a mammalian face can be categorized into regions associated with unique thermal profiles, with the following representative examples of approximate facial regions:
1) Forehead
2) Eyebrows
3) Bridge of nose
4) Nose
5) Temples
6) Eyes
7) Cheeks
8) Chin
9) Mouth
10) Upper lip,

Where the numbered facial regions above correspond to the numbered areas in FIG. 1.

### Schematic of Invention Workflow

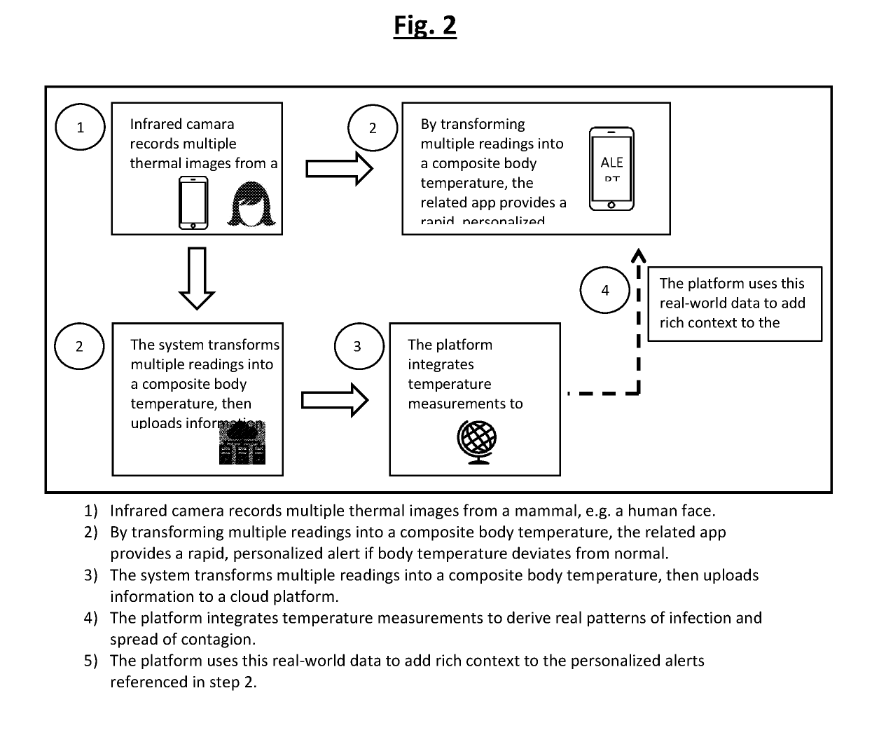

By reference to FIG. 2, a graphical schematic is provided by which the bidirectional flow of data occurs. The 5 distinct but interrelated steps, corresponding to numbers 1-5 in the graphical schematic, are identified below:
1) Infrared camera records multiple thermal images from a mammal, e.g. a human face.
2) By transforming multiple readings into a composite body temperature, the related app provides a rapid, personalized alert if body temperature deviates from normal.
3) The system transforms multiple readings into a composite body temperature, then uploads information to a cloud platform, where “the cloud” refers to on-demand delivery of compute power, database storage, applications, and other IT resources via the internet.
4) The cloud platform integrates temperature measurements to derive actual patterns of infection and spread of contagion.
5) The cloud platform uses this real-world data to add rich context to the personalized alerts referenced in step 2.

### SUMMARY OF INVENTION

### Individual-Level Temperature Monitoring

Electronic devices such as smartphones, laptops, tablet computers, smart televisions, etc. are often adapted to include sensors including cameras. Such cameras may be adapted to take thermal images including the thermal image of mammals such as by way of example, humans and cattle.

Normal human body temperature is known to fall approximately within the range of 36.5-37.5° C. (97.7-99.5° F.). Currently, body temperature can be measured rapidly by infrared cameras or digital thermometers (which may also use infrared sensors). These are usually stand-alone devices that, in some cases, can be connected to a smartphone (either by physical cable or via wireless communication protocol) which then records and uploads the temperature measurement. However, the requirements for (1) a standalone measurement device, and (2) a step of connecting it to a smartphone, are not conducive to measuring temperature rapidly and frequently throughout a given day or night. Therefore, they have limited utility toward identifying the specific timeline of fever onset. Conversely, our approach leverages the existing integration of infrared imaging hardware within the device, especially a smartphone, which facilitates frequent interaction between the user and the temperature-measuring application. For example, each time the user deploys infrared facial recognition to unlock the smartphone, body temperature readings would be collected automatically and simultaneously. Because consumers interact with their smartphone devices on average 80 or more times a day (New York Post, 2017), this drives the collection of sufficient daily data points to capture not only the user's baseline “healthy” temperature but also the early onset of fever. Taking by way of example the Apple iPhone's Face ID, this system projects 30,000 infrared points onto the user's face, then constructs an infrared image from these data. Each of these infrared points may be mapped against specific regions of the user's face [SEE FIG. 1], because specific regions of the face are known to reflect the user's body temperature in distinct ways. For example, extremities like the nose and the ears are known to have lower temperature profiles compared to capillary-rich facial regions like the forehead and cheeks. This approach is far more robust than the aforementioned single-point infrared measurements taken at the forehead.

Ultimately, each device would run this unsupervised deep learning exercise to construct an accurate, data-driven temperature profile that serves as a personalized baseline of its owner, against which future infrared measurements of that owner would be compared. To increase the clinical relevance of this deep learning exercise, these datasets at first could also be compared and/or trained against measurements obtained with sensitive, traditional thermometers (e.g. rectal, inguinal, axillary). Ultimately, however, the accuracy of the deep learning model should circumvent the need for training against traditional thermometer benchmarks, and the elimination of traditional thermometers further increases the likelihood of an owner engaging frequently with his/her temperature measurement device.

Thermal images of an individual mammal such as a human may vary depending on the body temperature of the individual at the time the thermal image is taken; therefore, a thermal image can be correlated with the temperature of the individual. An individual with an elevated body temperature above 99.5° C. will have a different thermal image than when the individual has a normal body temperature in the range of approximately 97.7° C.-99.5° C. The fact that baseline “normal” temperature exhibits natural variation among humans (U.S. National Library of Medicine, 2018) underscores the value of having each device trained specifically against the unique temperature profile of its owner. Elevated body temperature may be associated with fever, ovulation, heat stress due to exertion, certain cardiac conditions, and other conditions described below. The change in thermal image can be used to correlate to elevated temperature, and therefore predict and detect fever or other physical condition. As a supervised machine learning exercise, the range of biologically “normal” human body temperature would be contrasted against the range of temperatures that correspond to fever. Thermal images also show variation if an individual's body temperature is below the normal range such as when the individual is suffering from hypothermia, congestive heart failure, or other conditions. At the individual level, the invention provides a person with real-time information on changes in temperature in the context of possible harmful conditions such as fever, heat stress, etc.

### Community-Level Temperature Integration and Modeling

It is appreciated that electronic devices that contain sensors and cameras can also contain GPS elements. Therefore, thermal images that are captured by an electronic device can also be “geotagged” (the process of adding geographical identification metadata to an existing piece of data). Because thermal images can be correlated to temperature, and can also be geotagged, they have unique value as real-time, individual-level temperature data points that can be integrated into a large cloud-computing framework that tracks and analyzes actual incidences and patterns of disease outbreak. It is appreciated that electronic devices, especially smartphones, can also metatag thermal images with real-time, location-specific indicators of environmental factors such as temperature, humidity, elevation, climate, etc. The integration of these multiple real-time variables via deep learning potentially offers a far richer and more accurate assessment of influenza risk as compared to existing mathematical modeling approaches that rely on combinations of historical data, algorithm-generated estimates, or inferences based on online search engine results or location-based news reporting.

At the community level, the invention therefore provides a novel way to integrate individual-level data into a large, population-level computational model that can accurately track and predict migration patterns of infection-causing pathogens based on actual data points.

### Description of the Community-Level Temperature Integration Invention [See FIG. 2]

The integration of individual-level datapoints into a large dataset uncovers several potential applications, as outlined below.
1) Body temperatures will be recorded passively as outlined in the Individual-Level temperature monitoring section. Because normal or reference temperatures will have been obtained for each individual, personalized temperature thresholds can be established. Temperatures are uploaded to a community-level computational framework and are tagged as normal or abnormal along with geocoding and other metadata such as temperature, humidity, elevation, climate, etc.
2) The computational framework, using deep learning approaches (for example, unsupervised approaches), integrates these individually-generated temperature measurements into a dynamic map that traces and predicts severity, location, kinetics, and migration of fever. As individuals commute or travel great distances, the computational framework accounts for these changes and integrates them into potential infection transmission patterns which are of clear value to national and global health organizations. This is a novel, optimal method for epidemiological monitoring that incorporates actual fever, environmental, and travel-related data points.
3) The framework would also deliver safety notifications to vulnerable populations. For example, if epidemiological monitoring indicates that a city is highly vulnerable to an outbreak, resources such as flu vaccines, flu treatments, and extra nursing staff in that city should be deployed towards hospitals, nursing homes, daycare, etc. A digital diagnostic product that helps identify and stratify individuals according to their likely benefit from a flu vaccine and/or flu medication is an example of a “companion diagnostic”, which is a popular tool by which pharmaceutical companies determine whether a therapeutic drug is suitable for a specific person and/or population. A companion diagnostic is a potential use case for this invention. Whereas current companion diagnostics make recommendations based only on test results from an individual, the proposed invention would integrate individual-level fever data along with societal and environmental inputs to generate a more holistic and comprehensive score related to, by way of example, the likely benefit of receiving flu medication early in the course of flu infection.
4) Trends and risk patterns generated by this computational framework can also be transmitted as actionable recommendations back to individuals. Examples of possible transmissions from computational framework to individual:
   a) The individual's fever is increasing rapidly, and the framework recommends that he/she should visit a doctor. The invention, via a permissions-based data disclosure hierarchy, could also be authorized to transmit the individual's fever history to the doctor's office in advance of the visit.
   b) The individual lives or works in a region that has been designated as a possible hotspot for an outbreak of one or more pathogens, and the framework recommends that he/she take extra precaution at home and/or work.
   c) The individual has scheduled travel to a region that has been designated as a possible infection hotspot, and the framework recommends postponing or adjusting his/her travel. Digital virtual assistants, for example Apple's Siri, can be leveraged to convey personalized risk recommendations back to users in an actionable and intuitive way since virtual assistants are commonly synchronized to a user's travel and/or work schedule.
   d) Senior citizens are expected to be highly susceptible to a particular season's predominant influenza strains, and the framework recommends that they receive the flu shot early in the season.

### Additional Fever-Measurement Applications for Human Conditions

Hypothermia: this is the condition of the body dissipating heat and reaching temperatures below 35.0° C. (95.0° F.). Hypothermia has various causes including, but not limited to, drowning, skin disorders, burns, drugs, environmental conditions, medical interventions, and metabolic or neurological conditions. It is a potential cause of cardiac arrest, confusion, lethargy, loss of consciousness, coma, and death. Therefore, our rapid method of detecting abnormal temperature may provide critical real-time guidance to healthcare staff such as emergency medical technicians (EMTs), nurses, doctors, etc. when confronted with a possible case of hypothermia, especially early-stage hypothermia. In this use case, a device would not necessarily have to refer to a patient's personal baseline, because detection of the hypothermia threshold of 95.0° F. or below is of clinical value for any human regardless of their baseline temperature. A single device therefore could be used for multiple patients, because it would not be restricted to hypothermia evaluation in just one person.
Stress: this is a complex condition involving hormones, altered heart rate, activation of the nervous system, etc. Fever is a known outcome of certain kinds of stress. Our fever-monitoring invention can therefore serve as a useful adjunct to digital apps and services that are already attempting to track and mitigate stress as a component of mental and emotional wellness. For example, an emotional wellness app may encourage an individual to face toward the device (smartphone) camera, which can track and record the individual's temperature while also projecting words, images, haptic feedback, and/or sounds that promote wellness and happiness during times of stress.
Fertility: the fertility industry supports people that want to improve their success rate with either natural conception methods or in vitro fertilization procedures. Robust monitoring of ovulation cycles, which helps identify the precise window of ovulation, is a critical component of improving the likelihood of achieving pregnancy. Because temperature is correlated with ovulation, our invention would provide a real-time benefit for the synchronizing of ovulation with fertilization in the example of natural conception, and would also help with scheduling of egg retrieval in the case of in vitro fertilization.
Heatstroke: This condition is caused by the body overheating, usually as a result of prolonged heat exposure or physical exertion in high temperatures (for example, strenuous exercise in hot climates, or firefighters returning from a fire). Heatstroke requires emergency treatment and if left untreated can quickly damage the brain, heart, kidneys and muscles (Mayo Clinic, 2018). The damage is exacerbated by delays in treatment, increasing risk of serious complications or death, and emphasizes the value of rapid, real-time determination of heatstroke. A single device would not be restricted to evaluation of heatstroke in just one person since the clinical threshold is relatively universal; a single device could therefore be used for multiple people.

### Additional Nonhuman Applications

Ovulation in animals: The successful breeding of domesticated mammals is a critical component of food security for humans. For mammalian species including, for example, cattle, pigs, and sheep, ovulation detection/prediction enables the optimal scheduling of breeding techniques including (but not limited to) Artificial Insemination, Superovulation, In vitro Fertilization, and Embryo Transfer. In this application the infrared camera sensor would be directed toward the female's vaginal area to measure cyclical changes in vulvar temperature associated with estrus, the period of sexual receptivity and fertility in many female mammals (Sakatani, Takahashi, & Takenouchi, 2016). This would enable both veterinarians and non-veterinarians to rapidly and objectively categorize female animals by stage of estrus. Compared to previous applications of infrared thermography for animal estrus (Scolari, Clark, & Knox, 2011), the described invention offers a much greater level of insight because (1) it can rapidly integrate vulvar temperature along with environmental factors, (2) it can complete calculations within the on-site device itself instead of transmitting data to a separate off-site computer, and (3) it can simultaneously generate predictions and recommendations regarding animal infections with fever components. Furthermore, the prevalence of low-cost transmitter tags in the livestock industry (for example RFID ear tags) enables the automatic synchronizing of each infrared measurement to a specific animal, thereby expediting the collection and organization of animal temperature data.

### REFERENCES

Apple. (2018). About Face ID advanced technology. Retrieved from Apple Support:
https://support.apple.com/en-ca/HT208108

Berksoy, E., Bağ, O., Yazici, S., & Çelik, T. (2018, February). Use of noncontact infrared thermography to measure temperature in children in a triage room. Medicine (Baltimore), e9737.

Ioannou, S., Morris, P., Mercer, H., Baker, M., Gallese, V., & Reddy, V. (2014 Aug. 4).
Proximity and gaze influences facial temperature: a thermal infrared imaging study. Front. Psychol.

Mayo Clinic. (2018). Heatstroke. Retrieved from Mayo Clinic:
https://www.mayoclinic.org/diseases-conditions/heat-stroke/symptoms-causes/syc-20353581

New York Post. (2017 Nov 8). Americans check their phones 80 times a day.

Priest, P., Duncan, A., Jennings, L., & Baker, M. (2011). Thermal Image Scanning for Influenza Border Screening: Results of an Airport Screening Study. PLoS One, 6(1), e14490.

Sakatani, M., Takahashi, M., & Takenouchi, N. (2016). The efficiency of vaginal temperature measurement for detection of estrus in Japanese Black cows. J Reprod Dev, 62(2), 201-207.

Scolari, S., Clark, S., & Knox, R. (2011). Vulvar skin temperature changes significantly during estrus in swine as determined by digital infrared thermography. J Swine Health Prod., 19(3), 151-155.

Sun, L. (2017 Oct. 25). Drop in adult flu vaccinations may be factor in last season's record-breaking deaths, illnesses. The Washington Post. Retrieved from
https://www.washingtonpost.com/health/2018/10/25/drop-adult-flu-vaccinations-may-be-factor-last-seasons-record-breaking-deaths-illnesses/noredirect=on&utm_term=.ccf1ce80a9fd

U.S. National Library of Medicine. (2018, October). Body temperature norms. Retrieved from Medline Plus: https://medlineplus.gov/ency/article/001982.htm

Wagner, M. (2013, Oct. 17). Emerging Biometric Technology: Infrared Facial Matching. Retrieved from Dev Technology Group: http://devtechnology.com/2013/10/emerging-biometric-technology-infrared-facial-matching/

1. A method of detecting a change in body condition in a mammal comprising obtaining a first reference thermal image of the body or a part of the body of said mammal using a thermal imaging device when the mammal is within its normal body temperature range and does not have any symptoms or characteristics of illness or physical condition associated with body temperature different from its normal body temperature range; taking a second thermal image of the body or a part of the body of said mammal at a later time; comparing the intensity of the second thermal image to the first reference thermal image; and in response to such comparison, determining whether said mammal shows symptoms of a certain illness or physical condition associated with a change in body temperature and intensity of the thermal image; and displaying information regarding said determination on the first display of the first electronic device.

2. The method of claim 1 wherein the thermal imaging device is a smart phone or tablet having a camera with the capability to take thermal images.

3. The method of claim 1 wherein said mammal is a human.

4. The method of claim 3 wherein the part of the body for which the first reference thermal image and the second thermal image are obtained is of the face of said human.

5. The method of claim 3 wherein the illness or physical condition associated with a change in said human's body temperature is flu, stress, heatstroke, ovulation, hypothermia, or cardiac dysfunction.

6. The method of claim 1 wherein said mammal is a domesticated animal.

7. The method of claim 4 wherein the illness or physical condition associated with a change in said human's body temperature is flu, stress, heatstroke, hypothermia, or cardiac dysfunction.

8. A method of determining the body temperature of a mammal comprising obtaining thousands of infrared datapoints of said mammal's body or part of the body using thermal imaging, and using a deep learning engine to correlate these datapoints and thermal imaging to determine a single surrogate temperature value.
